(19) United States
(12) Patent Application Publication
(10) Pub. No.: US 2005/0085444 A1
(43) Pub. Date: Apr. 21, 2005

(54) Cosmetic or dermatological use of vitamin a or the esters thereof in combination with a partly methylated beta-cyclodextrin

(76) Inventors: Jean-Christophe Archambault, Meung Sur Loire (FR); Francoise Ouvrard-Baraton, Orleans (FR); Florence Djedaini-Pilard, Amiens (FR); Sandrine Weisse, Rennes (FR)

Correspondence Address: NATH & ASSOCIATES, 1030 15th STREET, NW, 6TH FLOOR, WASHINGTON, DC, 20005, US

(21) Appl. No.: 10/502,712

(22) PCT Filed: Jan. 31, 2003
(86) PCT No.: PCT/FR03/00294

(30) Foreign Application Priority Data
Feb. 1, 2002 (FR) 02/201241

(51) Int. Cl. A61K 31/724

(52) U.S. Cl. 514/58

(57) ABSTRACT

The invention relates to the use of dimethylated β-cyclodextrins, particularly DIMEB and RAMEB, as agents for promoting the penetration of vitamin A or its cosmetically or dermatologically acceptable esters into the epidermis, and their retention in the skin, for the preparation of a cosmetic or dermatological composition containing a combination of vitamin A or at least one of its cosmetically or dermatologically acceptable esters with said dimethylated β-cyclodextrins. It further relates to novel cosmetic or dermatological compositions containing these combinations of dimethylated β-cyclodextrins and vitamin A or vitamin A esters, particularly in the form of inclusion complexes. It further relates to uses of these compositions in the field of cosmetics or dermatology.

The present invention relates to the use of combinations of partially methylated β-cyclodextrins and vitamin A or vitamin A esters in the field of cosmetics and dermatology.

For many years, vitamin A and its esters, especially the acetate and palmitate, have been very widely used in cosmetics and dermatology for improving the general condition of the skin. The actions of vitamin A are well known in these fields of application. In particular, it regulates the cell metabolism of the skin and hence preserves or restores a good physiological skin condition. Thus vitamin A makes it possible to improve the tone, firmness and elasticity of the skin, which tend to decrease with age or due to the effects of stress. In particular, vitamin A or its esters are commonly used for delaying the appearance of wrinkles or reducing their depth. In dermatology, vitamin A acts effectively to promote healing of the skin and in the treatment of skin affected by acne.

However, the use of such compounds is limited in practice by problems of stability, solubility in aqueous media and also skin penetration. In fact, the use of vitamin A or its esters is of value in cosmetics and dermatology only if these active ingredients are bioavailable to the dermis and/or epidermis.

The technical problems encountered in achieving their solubilization, stabilization and skin penetration have therefore formed the subject of numerous developments. Methods have been described for solubilizing vitamin A or its esters in aqueous media and stabilizing them. However, the results obtained are unsatisfactory in terms of penetration into the deeper layers of the epidermis, there being a corresponding reduction in the bioavailability of the active ingredients incorporated in the cosmetic and dermatological compositions of the prior art.

α-, β- and γ-cyclodextrins, hereafter denoted by α-CD, β-CD and γ-CD, have long been described as vectors for all kinds of active ingredient, being capable of trapping certain small molecules of a hydrophobic nature. In particular, cyclodextrins are described as capturing and protecting lipophilic vitamins and flavorings.

Document WO 9421225 of the prior art discloses cosmetic compositions that use a β-cyclodextrin as a penetrating agent to form inclusion complexes of vitamin A esters, particularly the palmitate.

However, there is a major disadvantage in using β-cyclodextrin to produce this inclusion. In fact, it is found that vitamin A included in β-cyclodextrin is not protected and degrades even more rapidly than non-included vitamin A. This phenomenon was proved by Karl-Heinz Frömming et al. (Acta Pharm. Technol. (1988) 34 (3) 152-155) using various techniques, namely UV spectrophotometry, NMR spectroscopy, etc. Thus the use of β-cyclodextrin does not seem to be capable of solving all the technical problems referred to above for the cosmetic or dermatological use of vitamin A or its esters.

To improve the stability of vitamin A or its esters, it has been proposed to use γ-cyclodextrin as a host molecule (U.S. Pat. No. 5,985,296). However, the inclusion complex of vitamin A in γ-cyclodextrin has a very mediocre solubility in aqueous media, seriously limiting its value for a cosmetic or dermatological application.

It is also known, from document EP 0649653 A1, to use partially methylated β-CD as absorption promoters in pharmaceutical compositions for the percutaneous administration of active principles.

Said document manifestly does not encourage those skilled in the art to use a partially methylated β-cyclodextrin as a vector for the penetration of active principles into the skin without percutaneous passage, with a view to cosmetic applications.

Furthermore, patent application FR 2484252 describes a process for the preparation of aqueous solutions of biologically active organic compounds that are insoluble or sparingly soluble in water, such as vitamin A (or retinol) and its esters, by inclusion in partially methylated, particularly dimethylated, β-cyclodextrins. Said document states that these aqueous solutions are stable. By way of example, a process is described for the inclusion of vitamin A acetate in heptakis(2,6-di-O-methyl)-β-cyclodextrin. However, the inclusion complex formed by this process is then incorporated into a pharmaceutical preparation for oral administration. Said document encourages those skilled in the art to administer liposoluble vitamins, like vitamin A, orally in the form of aqueous solutions, thereby discouraging topical use.

Other authors have prepared inclusion complexes of different retinoids and their esters in cyclodextrins, particularly 2,6-di-O-methyl-β-cyclodextrin, or “DM-β-CD”, in order to carry out water solubility studies and stability studies (S. Muñoz Botella et al., Analyst (1996) 121, 1557-1560). Inclusion complexes with retinol and retinyl acetate in DM-β-CD have been prepared and studied. Observations by the fluorescence technique have shown that these complexes have an excellent long-term stability.

Finally, document U.S. Pat. No. 5,484,816 describes the preparation of a skin treatment composition in which the stability of the vitamin A is greatly improved. This positive effect on the stability is obtained especially by the inclusion of vitamin A or its ester in different modified or unmodified cyclodextrins, particularly a methyl-β-cyclodextrin.

The inventors of the present invention have now discovered, totally surprisingly, that the use of certain well-defined, partially methylated β-cyclodextrins, in combination with vitamin A or one of its esters conventionally used in cosmetics and dermatology, makes it possible to prepare cosmetic or dermatological compositions whose application affords a remarkable retention of the vitamin A or its esters in the skin, while avoiding their transdermic passage.

Thus the present invention results from the particularly surprising finding that no transdermic passage of the vitamin A or its ester took place when the vitamin A or its ester was combined in the composition with well-defined, partially methylated β-cyclodextrins.

This discovery proves particularly advantageous because it enables all the biological activity of the composition to be focused on the release of the active ingredient into the skin, which is the target zone according to the present invention.

The present invention thus relates to novel uses, in the field of cosmetics and dermatology, of combinations of well-defined, partially methylated β-cyclodextrins with vitamin A or vitamin A esters.

The invention further relates to novel cosmetic or dermatological compositions containing these combinations.

Before embarking on the actual detailed description of the invention, it seems appropriate to give a number of definitions of terms employed throughout the present description.

Cyclodextrins (CD) are cyclic compounds, also called cycloamyloses or cycloglucans, that consist of a concatenation of glucose units (6 glucose units for α-cyclodextrin, 7 for β-cyclodextrin and 8 for γ-cyclodextrin).

“Dimethylated β-cyclodextrins” are understood in terms of the invention as meaning methylated β-cyclodextrin derivatives that possess a methoxy group in, on average, two of the three positions 2, 3 and 6 of each of the 7 glucose units, the methoxy groups preferably being located in positions 2 and 6, and position 3 carrying a hydroxyl group. Statistically the “dimethylated β-cyclodextrins” according to the invention have a degree of substitution of the hydroxyl group protons of the glucose units by methyl radicals of between 10 and 16 and preferably of between 11 and 15.

In this family of dimethylated β-cyclodextrins, the following will be singled out in particular:
the dimethylated derivative in which all the glucose units possess a methoxy group in each of positions 2 and 6, i.e. which has a degree of substitution of 14 as defined above, said derivative being called heptakis(2,6-di-O-methyl)-β-cyclodextrin or DIMEB, and
among the commercial products existing in the family of dimethylated β-cyclodextrins, RAMEB. This is a β-cyclodextrin in which each glucose unit is randomly dimethylated, the mean degree of substitution of RAMEB by methyl radicals being about 12.6, as indicated especially in document WO 0145615.

According to a first essential characteristic, the present invention relates to the use of the methylated β-cyclodextrin derivatives called dimethylated β-cyclodextrins which possess a methoxy group in, on average, two of the three positions 2, 3 and 6 of each of the 7 glucose units, the methoxy groups preferably being located in positions 2 and 6, and position 3 carrying a hydroxyl group, and which statistically have a degree of substitution of the hydroxyl group protons of the glucose units by methyl radicals of between 10 and 16 and preferably of between 11 and 15, as agents for promoting the penetration of vitamin A or its cosmetically or dermatologically acceptable esters into the epidermis, and their retention in the skin, for the preparation of a cosmetic or dermatological composition containing a combination of vitamin A or at least one of its cosmetically or dermatologically acceptable esters with said dimethylated β-cyclodextrins.

According to a second essential characteristic, the invention relates to a cosmetic or dermatological composition for topical application to the skin, characterized in that it is in the form of a gel, a lotion, an emulsion or a powder and in that it contains, as the active ingredient, a combination of dimethylated β-cyclodextrins and vitamin A or at least one of its cosmetically or dermatologically acceptable esters.

According to a third characteristic, the invention relates to a method of cosmetic care.

More precisely, this is a method of cosmetic care that is intended especially to regulate the cell metabolism of the skin, and/or preserve or restore a good physiological skin condition, and/or improve the tone, firmness and elasticity of the skin, and/or delay the appearance of wrinkles or reduce their depth, and/or care for greasy skin, characterized in that it comprises the application to the skin of a cosmetic composition containing a combination of dimethylated β-cyclodextrins and vitamin A or at least one of its cosmetically acceptable esters.

The invention further relates to a method of dermatological treatment for improving the healing of the skin or treating skin prone to acne.

Thus, according to its last characteristic, the invention relates to the use of a combination of vitamin A or at least one of its dermatologically acceptable esters and a dimethylated β-cyclodextrin for the manufacture of a dermatological composition with healing activity or for the treatment of skin prone to acne.

As explained above, the present invention results from the fact that its inventors have demonstrated, as is apparent especially from the Examples below, that the use of a dimethylated β-cyclodextrin as defined above makes it possible greatly to improve the absorption of vitamin A or its esters by the skin, while avoiding transdermic passage, and thereby to increase its bioavailability.

The dimethylated β-cyclodextrins which can be used according to the invention in combination with vitamin A or one of its esters can be any β-cyclodextrins as defined above.

It is therefore possible, according to the present invention, to use any dimethylated β-cyclodextrin as defined above, these β-cyclodextrins having a degree of substitution of between 10 and 16 and preferably of between 11 and 15.

In one particularly advantageous variant, heptakis(2,6-di-O-methyl)-β-cyclodextrin, or DIMEB, will be used.

In another advantageous variant of the invention, the dimethylated β-cyclodextrins used will be β-cyclodextrins that are randomly dimethylated on each glucose and are commonly marketed under the name RAMEB, as defined above.

The invention may also be carried out with vitamin A itself in its alcohol form (retinol). However, a preferred choice will be to use its esters. All the vitamin A esters conventionally used in cosmetics and dermatology may be used according to the present invention. Vitamin A acetate and propionate will preferably be chosen.

As is already known in the literature, vitamin A and its esters easily form inclusion complexes with β-cyclodextrins, particularly with alkylated β-cyclodextrins such as DIMEB and RAMEB.

These inclusion complexes may be used to introduce the combinations of vitamin A or vitamin A esters and methylated β-cyclodextrins into the compositions of the invention; in general, vitamin A or its ester will be at least partially included in dimethylated β-cyclodextrin.

Thus, to prepare the inclusion complexes for carrying out the invention, it is possible to use one of the described processes, particularly the one described in document FR 2484252. In said process, dimethylated β-cyclodextrin is generally dissolved in water, then vitamin A or its ester is added to the resulting solution, either directly or in the form of a water-miscible organic solution, and then the solvent is evaporated off in order to recover the inclusion complex in solid form.

In another variant, the inclusion complex can also be recovered in solution before any evaporation, or alternatively the inclusion complex in solid form obtained in the previous step can be resolubilized in an aqueous medium for subsequent use within the framework of the present invention.

It is also possible to use the preparative processes described by S. Muñoz Botella et al. (Analyst (1996) 121, 1557-1560). For example, according to the principle of the first of these processes described in said publication, a solution of retinol in hexane is introduced into a round-bottom flask and the solvent is then evaporated off to form a thin film of retinol on the walls of the flask. An aqueous solution of dimethylated β-cyclodextrin is then introduced into the flask, after which this solution is stirred for 24 to 48 hours by magnetic means.

Such preparative processes are found to be particularly valuable because they make it possible to obtain aqueous solutions, if appropriate in gel form, of vitamin A and its esters.

According to one feature of the invention, the inclusion complexes defined above can be used either in the form of aqueous solutions or in the form of dry powders for incorporation into cosmetic or dermatological compositions.

It is also apparent that the cosmetic or dermatological composition according to the invention can be prepared in different forms, particularly in the form of an aqueous gel, a lotion, an emulsion or a powder.

In particular, these products will be cosmetic care products or cosmetic make-up/treatment products intended especially for combating the appearance of the effects of actinic or chronological ageing, such as wrinkles or the loss of skin elasticity and tonicity, or intended for the care of greasy skin.

They will also be dermatological preparations intended especially for promoting healing of the skin or for the treatment of skin prone to acne.

The concentrations of combined active ingredients contained in the cosmetic or dermatological compositions of the invention can vary within wide limits.

However, the compositions of the invention advantageously contain from 0.0001% to 0.5% by weight, preferably from 0.001% to 0.1% by weight, of vitamin A or its esters and from 0.0005% to 10% by weight, preferably from 0.005% to 1% by weight, of dimethylated β-cyclodextrin.

As explained earlier, the above two constituents are advantageously in the form of an inclusion complex, this inclusion complex advantageously being in a concentration of between 0.005% and 2.5% by weight, preferably of between 0.005% and 0.5% by weight, in these compositions.

In one advantageous variant, the proportion by weight of vitamin A or its esters relative to dimethylated β-cyclodextrin varies between 0.1 and 1.

The Examples which follow are given purely in order to illustrate the invention.

### EXAMPLES

Unless indicated otherwise, the concentrations are expressed in percentages by weight.

### Example 1

### Preparation of an Aqueous Gel Containing an Inclusion Complex of Vitamin A Propionate in RAMEB (or Commercial dimethyl-β-cyclodextrin)

The vitamin A ester used is vitamin A propionate, hereafter denoted by VAP.

200 μl of a 1 M acetone solution of VAP are added to 10 ml of a 50 mM solution of RAMEB to give a final VAP concentration of 20 mM.

The mixture is stirred under a ventilated hood for 24 hours.

The solutions obtained are then centrifuged for 6 minutes at 6000 rpm. The solution obtained after this operation is clear and pale yellow in color and does not contain a precipitate.

The aqueous phase is filtered, frozen and lyophilized.

This gives a powder, which can be used in this form in the preparation of a cosmetic or dermatological composition or, if required, can easily be redissolved in an aqueous medium.

The above-mentioned aqueous medium can be an aqueous gel, for example a 1.8% gel of hydroxyethyl cellulose (Natrosol®, Hercules Inc., USA) in water.

In the present Example, 3.3 g (25%) of an aqueous solution of the inclusion complex prepared above in powder form are added to 10 g (75%) of aqueous hydroxyethyl cellulose gel defined above, this corresponding to an amount of 24 mg of VAP (i.e. a final concentration of about 0.72% of VAP). This gives a gel containing the inclusion complex of VAP in RAMEB which can be used according to the invention.

### Example 2

### Penetration of the VAP into the Epidermis

The aim of this study is to compare the penetration and distribution of VAP in fresh human epidermis according to whether it is in the form of an inclusion complex with a dimethyl-β-cyclodextrin or with a γ-cyclodextrin, as well as incorporated in a gel in the absence of any cyclodextrin.

1. Preparation of the Gels

The following gels are prepared:

a) Base Gel

This gel consists of 1.8% by weight of hydroxyethyl cellulose (Natrosol®, Hercules Inc., USA) in water.

b) Gel Containing the RAMEB/VAP Inclusion Complex Which can be Used According to the Invention (RAMEB/VAP Gel)

The RAMEB gel is the gel prepared in Example 1 above.

c) Comparative Gel Containing the Inclusion Complex of VAP and β-cyclo-dextrin (Gamma/VAP Gel)

The gamma/VAP gel consists of 10 g (75%) of base gel to which are added 3 ml (25%) of an aqueous suspension containing 32 mg/ml of γ-CD/VAP inclusion complex prepared as indicated below, i.e. a theoretical amount of 24 mg of VAP (or a final VAP concentration of about 0.72%).

The γ-CD/VAP inclusion complex is prepared as follows: 200 μl of a 1 M acetone solution of vitamin A propionate (final concentration 20 mM) are added to a 10 mM solution of γ-CD. The mixture is stirred under a ventilated hood (to evaporate the acetone) for 24 h. The solutions obtained are then centrifuged for 6 minutes at 6000 rpm. The precipitate obtained is washed by centrifugation with 2×3 ml of water to remove the free CD and 2×3 ml of ether to remove the free vitamin A propionate, and is then dried under a stream of nitrogen, frozen and lyophilized.

d) Control/VAP Gel

The control/VAP gel consists of 25 g of base gel (75%) to which are added firstly a mixture of 60 mg of VAP (i.e. a final VAP concentration of about 0.72%) and 0.83 g of Tween 60 (polyethylene (20) sorbitan monostearate) (i.e. a final Tween 60 concentration of 2.5%) and then 7.5 g of water.

The study of penetration into the epidermis is conducted using a Franz cell having a surface area of 2 cm² and a volume of 4.2 ml. The reception liquid consists of commercial phosphate buffer (Sigma) (10 nM, NaCl 120 mM, KCl 2.7 mM, pH 7.4), commercial 0.1% sodium nitride (Sigma), 20% ethanol and 4% LRI solubilizer (commercially available mixture of polypropoxylated/polyethoxylated butyl ether and polyethylenated hydrogenated castor oil).

15 samples of epidermis are then prepared from 5 skin fragments of different origin, cut into three, from which the dermis has been separated beforehand under the action of heat according to a conventional technique. These samples are mounted on 15 Franz cells. In the course of one and the same experiment the 3 gels (RAMEB/VAP, gamma/VAP and control/VAP) are applied occlusively to the samples, each gel being applied to five of said samples.

After a contact time of 24 hours, the excess gel on the surface of the skin is washed off with the aid of cotton buds. The cotton buds together with the upper part of the cell are immersed in 10 ml of isopropanol and ultrasonicated for 15 min and then stirred for 2 hours. The solutions are diluted to 1/10 and filtered on a 0.2 μm filter before assaying. The stratum corneum is removed onto sticky patches by the stripping technique using an apparatus described in document FR 2710517, Parfums Christian Dior. Seven successive removals are made per sample (pressure 300 g/cm², duration 12 s). The sticky patches corresponding to each sample are placed in 2 ml of methanol overnight at 4° C. to extract the VAP. The methanolic medium is subsequently stirred for 2 hours and then filtered on a 0.2 μm filter before assaying. The epidermis with the stratum corneum removed is in turn placed in 1 ml of methanol to extract the VAP by the same procedure as above.

The VAP assays are performed by a conventional HPLC method.

The results of the VAP assays, in the stratum corneum, epidermis and reception liquid respectively, are collated in the Table below, in which the values are expressed as the percentage of VAP extracted relative to the amount of VAP applied to the sample.

| | stratum corneum | epidermis | reception liquid |
|---|---|---|---|
| control gel | 0.378 | 0.139 | 0.000 |
| RAMEB gel | 0.612 | 0.516 | 0.003 |
| gamma gel | 0.187 | 0.068 | 0.000 |

It is seen that the percentage of VAP which has penetrated the skin is much higher for the RAMEB/VAP gel, irrespective of the compartment in question. The gel obtained from the γ-CD/VAP complex is less effective than the control gel. For the RAMEB/VAP gel, it is again seen that the percentage of VAP in the epidermis is of the same order of magnitude as that in the stratum corneum, representing a high efficacy in terms of penetration. On the other hand, for the other two gels, the percentage of VAP in the epidermis is much lower.

### Example 3

### Influence of the Control

A study identical to that described in Example 2 is carried out with the RAMEB/VAP gel and VAP on its own in an oily medium.

The oily medium is a commercial carnation oil containing 0.75% of VAP. The results of the VAP assay in the different skin compartments show that ten times the amount of VAP is found in the reception liquid when it is in the form of a RAMEB/VAP inclusion complex. It can therefore be concluded that the RAMEB/VAP gel allows excellent penetration of the VAP into the epidermis in comparison with VAP on its own, irrespective of the formulation of the latter.

### Example 4

### Penetration of the VAP into Human Skin

The purpose of this study, carried out on fresh human skin, is to compare the penetration and distribution of the VAP across the human cutaneous integument (stratum corneum, epidermis and dermis) as a function of the gel.

The gels used are the control/VAP gel and the RAMEB/VAP gel as described in Example 2.

The study is carried out using the Franz cells characterized above.

The reception liquid has the same composition as that used in Example 2.

In the course of one and the same experiment the 2 gels are applied, by the same method as in Example 2, to 24 samples originating from 8 skin fragments of different origin.

After a contact time of 24 hours, the excess gel on the surface of the skin is washed off with the aid of cotton buds.

The cotton buds together with the upper part of the cell are immersed in 10 ml of isopropanol and ultrasonicated for 15 minutes and then stirred for 2 hours.

The solutions are diluted to 1/10 and filtered on a 0.2 μm filter before assaying.

The stratum corneum is collected by 7 successive strippings (pressure 300 g/cm², duration 12 s). The VAP is extracted by solubilization in 2 ml of methanol overnight at 4° C., followed by stirring for 2 hours and finally filtration on a 0.2 μm filter before assaying.

With the stratum corneum removed, the epidermis is separated from the dermis with the aid of a scalpel.

The VAP is extracted from the epidermis by solubilization of the latter in 1 ml of methanol overnight at 4° C., followed by stirring for 2 hours and finally filtration on a 0.2 μm filter before assaying.

The VAP assays are performed by HPLC.

The VAP was assayed in the different skin compartments. No trace of VAP was detected in the reception liquid, in contrast to the results obtained in Example 2.

The RAMEB/VAP gel does not therefore promote transdermic passage of the VAP.

### Example 5

### Dermatological and Cosmetic Compositions

The RAMEB/VAP complex prepared according to Example 1 is used as the active principle in the Examples of composition formulations according to the invention.

a) Dermatological healing gel

| | |
|---|---|
| Glycol | 3 |
| Sepigel 305 | 3 |
| Cremophor CO 60 solubilizer | 2 |
| PEG (Pluriol E 1505) | 1.5 |
| Preservative | 0.5 |
| Perfume | 0.3 |
| Water | 89.2 |
| RAMEB/VAP complex | 0.5 |

b) Antiwrinkle day cream (emulsion)

| | |
|---|---|
| Brij 721 | 2.5 |
| Tegin 90 | 1.1 |
| Stearyl alcohol | 5 |
| Glycerol tricaprate/caprylate | 20 |
| Butylene glycol | 3 |
| Glycerol | 2 |
| Preservative | 0.5 |
| Perfume | 0.5 |
| Water | 64.9 |
| RAMEB/VAP complex | 0.5 |

c) Revitalizing lotion

| | |
|---|---|
| Butylene glycol | 3 |
| EDTA | 0.1 |
| Solubilizer | 1 |
| Perfume | 0.3 |
| Alcohol | 5 |
| Water | 90.4 |
| RAMEB/VAP complex | 0.2 |

d) Powder for the care of greasy skin

| | |
|---|---|
| Talcum | 20 |
| Mica | 20 |
| Sericite | 20 |
| Pigments | 8 |
| Organic powder (nylon) | 20 |
| Silica | 8.75 |
| Mineral or silicone oil | 3 |
| RAMEB/VAP complex | 0.25 |

1-16. (canceled)

17. A method for promoting the penetration of an active ingredient selected from vitamin A and its cosmetically or dermatologically acceptable esters into the epidermis and its retention in the skin comprising the topic application of a cosmetic or dermatological composition containing said active ingredient and a methylated β-cyclodextrin derivative, called dimethyl β-cyclodextrin, said derivative possessing a methoxy group, in on average, two of the three positions 2, 3 and 6 of each of the 7 glucose units and statistically having a degree of substitution of the hydroxyl group protons of the glucose units by methyl radicals of between 10 and 16.

18. The method of claim 17, wherein said methoxy groups are located in positions 2 and 6 and position 3 carries a hydroxyl group.

19. The method according to claim 17, wherein said degree of substitution is comprised between 11 and 15.

20. The method according to claim 17, wherein said dimethylated cyclodextrin essentially consists of heptakis-2,6-di-O-methyl-β-cyclodextrin, also known as DIMEB.

21. The method according to claim 17, wherein said dimethylated cyclodextrin is essentially a β-cyclodextrin that is randomly dimethylated on each glucose unit with a degree of substitution in the order of 12.6, said dimethylated β-cyclodextrin commonly being known as RAMEB.

22. The method according to claim 17, wherein said vitamin A ester is selected from the group consisting of vitamin A acetate and propionate.

23. The method according to claim 17, wherein said active ingredient is introduced into the cosmetic composition in the form of an inclusion complex of said vitamin A or its ester with said dimethylated β-cyclodextrin.

24. The method according to claim 17, wherein said active ingredient is at least partially included in a dimethylated β-cyclodextrin.

25. The method according to claim 17, wherein said composition is in the form of a gel, a lotion, an emulsion or a powder.

26. The method according to claim 17, wherein said composition contains from 0.0001% to 0.5% by weight of vitamin A or one of its esters and from 0.0005% to 10% by weight, preferably from 0.005% to 1% by weight, of dimethylated β-cyclodextrin, the different percentages being expressed by weight based on the total weight of said composition.

27. The method according to claim 26, wherein said composition contains from 0.0005% to 2.5% by weight of an inclusion complex of vitamin A or its ester in dimethylated β-cyclodextrin.

28. A method of cosmetic or dermatological care intended to regulate the cell metabolism of the skin, and/or preserve or restore a good physiological skin condition, and/or improve the tone, firmness and elasticity of the skin, and/or delay the appearance of wrinkles or reduce their depth, and/or care for greasy skin, comprising the application to the skin of a cosmetic composition containing a combination of a dimethylated β-cyclodextrin and vitamin A or at least one of its cosmetically acceptable esters, the components of said combination being as defined in claim 17.
